(19) United States
(12) Patent Application Publication
(10) Pub. No.: US 2026/0069551 A1
(43) Pub. Date: Mar. 12, 2026

(54) Patch for treating demyelinating diseases

(71) Applicant: Johannes Gutenberg-Universität Mainz, Mainz (DE)

(72) Inventors: Claire Jacob, Mainz (DE); Peter Langguth, Mainz (DE); Jozef Al-Gousous, Mainz (DE); Sophia Engel, Wiesbaden (DE)

(21) Appl. No.: 19/109,598

(22) PCT Filed: Sep. 12, 2023
(86) PCT No.: PCT/EP2023/074981

§ 371 Date: Mar. 7, 2025

(30) Foreign Application Priority Data
Sep. 13, 2022 (EP) 22195291.4

(51) Int. Cl. A61K 9/70; A61K 9/00; A61K 31/522; A61K 47/10; A61K 47/38

(52) U.S. Cl. CPC A61K 9/7069; A61K 9/0014; A61K 31/522; A61K 47/10; A61K 47/38

(57) ABSTRACT

A pharmaceutical composition in the form of a transdermal patch comprising aminophylline or theophylline for use in the treatment or prevention of a hypomyelinating or a demyelinating disease or condition or a lesion of the peripheral or central nervous system where demyelination occurs. The patch may comprise a matrix layer comprising a matrix polymer, an active ingredient and a permeation enhancer.

### BACKGROUND

The present disclosure relates to the treatment or prevention of hypomyelinating or demyelinating diseases or conditions.

Demyelinating diseases belong to diseases of the nervous system in which the myelin sheath of neurons is damaged. This damage impairs the conduction of signals in the affected nerves. In turn, the reduction in conduction causes deficiency in sensation, movement, cognition, or other functions depending on which nerves are affected. The demyelinating disease usually includes diseases affecting the central nervous system and peripheral nervous system. Examples of such demyelinating diseases of the peripheral nervous system include Guillain-Barré syndrome and Charcot Marie Tooth (CMT) disease, whereas demyelinating diseases of the central nervous system include multiple sclerosis.

Demyelinating diseases may be caused by an overexpression of peripheral myelin protein 22 (PMP22). PMP22 is a protein which in humans is encoded by the PMP22 gene. The integral membrane protein encoded by this gene is a hydrophobic, tetraspan glycoprotein expressed mainly in Schwann cells and is a major component of compact myelin in the peripheral nervous system. Various mutations of the gene are causes of CMT1A. Dejerine-Scottas disease, and hereditary neuropathy with liability to pressure palsy (HNPP). Demyelinating lesions include, but are not limited to lesions of the nervous system myelin sheath, traumatic lesions of the nervous system, peripheral nerve injury or spinal cord injury.

The myelin sheath is a layer of lipid cell membrane that covers sheath nerve fiber axon outside and consists of myelin sheath cells, which main physiological function is to act as an insulation and protection functions on nerve axon, and facilitates rapid transmission of nervous impulse.

WO 2018/224650 suggests that hypo- and demyelinating diseases and conditions may be prevented or treated by the use of histone deacetylase 1 or 2 (HDAC1/2) activators, i.e. compounds that increase the expression or activity of HDAC1/2. For example, theophylline, a methylxanthine which is also well-known as phosphodiesterase inhibitor is proposed by WO 2018/224650 as a useful HDAC1/2 activator. The document shows the effectiveness of relatively high daily doses (10 mg/kg/day) of intraperitoneally injected theophylline in achieving significant remyelination of injured nerves. However, chronic treatment based on daily injections may not be very convenient to patients, and thus there is a need for providing alternative treatment methods which result in controlled systemic exposure to theophylline, and which are capable of achieving and maintaining effective plasma levels of the drug substance while being convenient to patients and health care providers, thereby increasing patient adherence and eventually improving therapeutic outcome.

U.S. Pat. No. 6,277,402 proposed the treatment of multiple sclerosis by administering a histamine H2 agonist in an amount which is effective to stimulate production of cyclic AMP. To further augment the effect of the H2 agonist, the document proposes to co-administer a phosphodiesterase inhibitor for the purpose of maintaining the level of cyclic AMP which is thus produced. According to the document, the preferred H2 agonist is histamine phosphate, and the preferred phosphodiesterase inhibitor is caffeine. Theophylline is mentioned as an alternative phosphodiesterase inhibitor. Moreover, the document proposes the transdermal delivery of the drug combination, and specifically discloses a patch of 6 mm diameter loaded with 0.2 mL of a gel containing 1.1 mg histamine diphosphate and 100 mg caffeine citrate, designed to be worn for 8 hours, but without providing any evidence of the actual extent of drug release or skin permeation. Importantly, U.S. Pat. No. 6,277,402 is entirely silent on the role of methylxanthines as HDAC1/2 activators, and does not recognise the potential of methylxanthines on their own as effective agents for treating demyelination.

US 2014/0276478 describes transdermal drug delivery systems for the administration of tertiary amine drugs which incorporate the drug substance in free base form within a polymer matrix which also contains a carboxylic group-containing compound with which the amine drug forms a salt in situ. The preferred drug is rivastigmine which is also the only exemplified drug, but the document also mentions a large number of other drugs with amino groups including theophylline for which the same general formulation principle is proposed. As a side note, it is doubtful that the formulation principle proposed is the document would actually work for theophylline, as theophylline is amphoteric and only weakly basic.

Against this background, it is the object of the present invention to provide an improved therapy for preventing or treating hypo- and demyelinating diseases and conditions. A further object is to enable the non-injectable systemic delivery of HDAC1/2 activators. Yet further objects include to provide treatment options that are suitable for chronic therapy, convenient to patients and health care providers, and to provide preventive and curative treatments for patients potentially affected by a demyelinating disease or condition which achieve significant plasma levels of HDAC1/2 activators such as methylxanthines, e.g. theophylline. Further objects addressed by the present disclosure will become clear on the basis of the following description including the examples and the claims.

### SUMMARY OF THE INVENTION

In a first aspect, a pharmaceutical composition is provided which comprises an active ingredient selected from aminophylline or theophylline or a salt thereof for use in the treatment or prevention of a hypomyelinating or demyelinating disease or condition, wherein the composition is in the form of a transdermal patch, and wherein the aminophylline or theophylline or salt thereof is the sole active ingredient in the transdermal patch.

In a further aspect, a transdermal patch is provided which comprises a matrix layer, wherein the matrix layer comprises at least 5 wt. % of an active ingredient selected from aminophylline or theophylline or a salt thereof. In some of the preferred embodiments, the active ingredient is aminophylline.

In yet a further aspect, a transdermal patch is provided which comprises (a) a backing layer; (b) a matrix layer comprising aminophylline, a matrix polymer, a plasticizer, and a permeation enhancer; and (c) a removable protective release liner; for use in the treatment or prevention of a hypomyelinating or a demyelinating disease or condition or a lesion of the peripheral or central nervous system where demyelination occurs.

The transdermal patch of the present invention allows a constant slow delivery of pharmaceutically or therapeutically effective amounts of aminophylline or theophylline, or a pharmaceutically active salt thereof. In some preferred embodiments, the transdermal patch comprises aminophylline, which is physiologically converted into theophylline in vivo. Theophylline can be measured in the plasma of blood samples taken from a human subject. A plasma concentration between 0.3 and 2.5 μg/ml is preferred in order to promote myelination and/or remyelination. In an alternative embodiment, the transdermal patch of the invention comprises theophylline, which is delivered continuously over a prolonged period of time, preferably at least 2 days, more preferably over a period of 4 consecutive days.

Other aspects are disclosed in the detailed description and the claims.

### BRIEF DESCRIPTION OF THE DRAWINGS

FIG. 1 is a simplified, schematic, cross-sectional view of a matrix-type transdermal patch (10).

FIG. 2 is a simplified, schematic, cross-sectional view of a reservoir-type transdermal patch (20).

FIG. 3 is a simplified, schematic, cross-sectional view of a matrix-type transdermal patch (30) with additional features for skin adhesion.

FIG. 4 is a simplified, schematic, cross-sectional view of a matrix-type transdermal patch (40) with alternative additional features for skin adhesion.

FIG. 5 shows the in vitro skin permeation of the active ingredient over time achieved by the transdermal patch prepared and tested according to Example 1.

### DETAILED DESCRIPTION OF THE INVENTION

According to a first aspect, the present disclosure provides a pharmaceutical composition which comprises an active ingredient selected from aminophylline or theophylline or a salt thereof for use in the treatment or prevention of a hypomyelinating or demyelinating disease or condition, wherein the composition is in the form of a transdermal patch, and wherein the aminophylline or theophylline or salt thereof is the sole active ingredient in the transdermal patch.

The inventors have found that unexpectedly high amounts of theophylline, as may be required in the therapy of demyelinating diseases, can actually be delivered systemically by the transdermal route. In other words, it was found that pharmaceutical compositions in the form of transdermal patches can incorporate and deliver significant amounts of drug through the skin, such as to enable effective treatment of demyelinating diseases, as will be further explained below. Moreover, as the inventors have recognised that aminophylline or theophylline are as such, when used at adequate dosages, biologically active agents that are themselves therapeutically effective in the prevention or treatment of demyelinating diseases. The composition therefore comprises only aminophylline or theophylline or a salt thereof as active ingredient.

As used herein, a pharmaceutical composition is any composition that is designed, formulated and processed such as to be adapted for use as a pharmaceutical product, and in particular for being administered to a human subject for the purpose of preventing, treating, preventing the deterioration of, or otherwise managing a disease or condition that may affect the subject. This may include certain considerations with respect to the selection of excipients, which should be safe and suitable for human use, and other features of the composition, which should be mostly compliant with generally accepted practices and regulatory guidances and regulations.

As said, the pharmaceutical composition is in the form of a transdermal patch. In this context, a transdermal patch should be understood as a film-, tape-, plaster- or patch-shaped product which is relatively flat, sufficiently flexible to be adapted for application to the skin of a subject, and which has the capability to adhere to the skin. Moreover, a transdermal patch is adapted to release and deliver an active ingredient through the skin to the bloodstream of a subject such as to make the active ingredient systemically bioavailable. This is, for example, in contrast to wart plasters which may comprise agents such as salicylic acid that are intended to act locally on the skin. Other expressions commonly used to describe transdermal patches are transdermal (drug) delivery systems (TDS) or transdermal therapeutic systems (TTS). In contrast to transdermal gels, transdermal patches deliver a specific drug dose to a subject via a defined area of skin, which is important to ensure that therapeutically effective systemic drug concentrations are achieved. In the present disclosure, the transdermal patch may also simply referred to as “patch”. Therefore, unless the context dictates otherwise, a reference to a patch should be understood as referring to the transdermal patch according to an aspect of the invention.

Demyelinating diseases are a group of disorders that affect the central nervous system (brain and spinal cord) and the peripheral nervous system by damaging the myelin sheath, a protective covering around nerve fibers. Myelin serves as an insulating layer, allowing nerve impulses to travel quickly and efficiently along the nerve fibers. When demyelination occurs, nerve conduction is disrupted, leading to various neurological symptoms. Some common demyelinating diseases include multiple sclerosis (MS), Guillain-Barré syndrome (GBS), neuromyelitis optica (NMO), and transverse myelitis. What these demyelinating diseases have in common is the involvement of the myelin sheath and the subsequent disruption of nerve signalling. While the specific causes and mechanisms may differ among these diseases, they share similar neurological symptoms such as weakness, loss of coordination, sensory disturbances, and problems with vision or speech. The course and severity of demyelinating diseases can also vary, ranging from acute and rapidly progressing conditions to chronic and relapsing-remitting forms.

Some common demyelinating diseases include multiple sclerosis (MS), Guillain-Barré syndrome (GBS), neuromyelitis optica (NMO), and transverse myelitis. MS is an autoimmune disease where the body's immune system mistakenly attacks the myelin in the central nervous system. This leads to inflammation, demyelination, and the formation of scar tissue (sclerosis) in multiple areas of the brain and spinal cord. The symptoms of MS vary widely and may include fatigue, muscle weakness, difficulty with coordination, visual disturbances, and cognitive problems. GBS is an acute inflammatory disorder that affects the peripheral nervous system. It is believed to be triggered by an infection or a prior illness. GBS causes the immune system to attack the myelin surrounding peripheral nerves, leading to weakness, paralysis, and in severe cases, respiratory failure. NMO, also known as Devic's disease, is an autoimmune disorder that primarily affects the optic nerves and the spinal cord. It leads to inflammation and demyelination in these areas, causing visual disturbances, weakness, and sensory loss. Transverse myelitis involves inflammation and demyelination of the spinal cord. It can be caused by infections, immune system disorders, or other unknown factors. Symptoms include motor and sensory deficits below the level of the spinal cord lesion.

Hypomyelinating disorders are a heterogeneous group of white matter disorders characterised by abnormally low amounts of myelination. Hypomyelinating disorders may be distinguished from other myelin disorders in that hypomyelination is typically a permanent deficiency in myelin deposition rather than myelin destruction (i.e. demyelination) or abnormal myelin deposition (i.e. dysmyelination).

In some of the preferred embodiments, the hypomyelinating or demyelinating disease or condition is selected from leukodystrophies, demyelinating diseases of the central nervous system, multiple sclerosis, central pontine myelinolysis, glioma, schizophrenia, demyelination due to aging, diabetes or due to toxic agents, chronic inflammatory demyelinating polyneuropathy (CIDP), Charcot-Marie-Tooth disease (CMT), Guillain-Barré syndrome, hereditary neuropathy with liability to pressure palsy (HNPP), progressive inflammatory neuropathy, Dejerine-Sottas disease, Waardenburg syndrome, congenital hypomyelinating neuropathy (CHN), Cowchock syndrome, Rosenberg-Chutorian syndrome, Roussy-Levy syndrome, lesion of the peripheral or central nervous system associated with demyelination, traumatic lesion of the nervous system, peripheral nerve injury or spinal cord injury.

As disclosed herein, the sole active ingredient in the transdermal patch is aminophylline or theophylline or a salt thereof. Since aminophylline is sometimes described as a salt of theophylline, the expression “or a salt thereof” should be understood as referring to another salt of theophylline which is not aminophylline. As will be discussed in more detail, the preferred active ingredient is selected from aminophylline and theophylline, and a particularly preferred active ingredient is aminophylline.

Theophylline, also known by its chemical names 1,3-dimethylxanthine and 1,3-dimethyl-7H-purine-2,6-dione, is a methylxanthine that has been used as a drug substance for decades, in particular for its pharmacological activity as a vasodilator, bronchodilator, phosphodiesterase inhibitor, and purinergic P1 receptor antagonist. Its currently approved therapeutic indications include the treatment of reversible airflow obstructions and exacerbations in lung diseases such as chronic obstructive pulmonary disease (COPD), asthma, and emphysema.

Aminophylline is a complex (sometimes also referred to as a salt) of two molecules of theophylline with one molecule of ethylenediamine, in pure form often provided as a dihydrate, but also available as anhydrate. In some preferred embodiments, anhydrous aminophylline is used as active ingredient in the transdermal patch. The chemical formula of aminophylline is usually recited as 1H-Purine-2, 6-dione, 3,7-dihydro-1,3-dimethyl-, compound with 1,2-ethanediamine (2:1). The molecular weight of aminophylline dihydrate is 456.46 g/mol. Accordingly, even when using aminophylline as the active ingredient in the transdermal patch, the pharmacologically active molecule in the body of the human subject to which the patch is administered is always theophylline. The same is true of any other salt or complex of theophylline that may be used as the active ingredient in the patch.

In a related aspect, the present disclosure provides a transdermal patch comprising a matrix layer, wherein the matrix layer comprises at least 5 wt % of an active ingredient selected from aminophylline or theophylline or a salt thereof. In some of the preferred embodiments relating to this aspect, the active ingredient is aminophylline. Other optional and preferred features are described below; these are applicable both to the inventive transdermal patch as such as well as to the pharmaceutical composition for use in the treatment or prevention of a hypomyelinating or demyelinating disease or condition.

As said, in some of the preferred embodiments, whether relating to the transdermal patch as such or to the inventive use of the patch, the active ingredient incorporated in the transdermal patch is aminophylline. The inventors have unexpectedly found that transdermal patches with aminophylline may achieve a particularly high permeation of the active ingredient, or at least of theophylline, through the skin such that effective theophylline plasma levels may be reached more easily, or with smaller patch sizes. This is rather surprising as the salt or complex form of the active ingredient with a substantially increased molecular weight would normally be expected to be associated with decreased skin permeation rates compared to theophylline, whose molecular weight is only 180.16 g/mol. To the knowledge of the inventors, it was found for the first time that theophylline may be delivered through the skin particularly effectively when used as aminophylline.

In some further embodiments, the transdermal patch is adapted to transdermally deliver a dose of at least about 20 mg of the active ingredient, calculated as theophylline, to a human subject within 24 hours. As mentioned, the inventors have found that high amounts of theophylline can be made systemically bioavailable via the transdermal delivery route. Based on the technical guidance provided herein, a person skilled in the art will have no difficulty in designing patches that deliver these amounts of theophylline. It should be noted that the dose, in this context, is calculated as theophylline, even if the active ingredient incorporated in the patch is aminophylline or another salt or complex of theophylline. Moreover, the expression “to transdermally deliver” means that the transdermal patch, when applied to the skin of a human subject, delivers the active ingredient in such a way that the specified dose reaches the systemic blood circulation. Also preferred are embodiments in which the patch is adapted to transdermally deliver a daily dose of at least about 30 mg, or at least about 50 mg, such as from about 50 mg to about 250 mg of the active ingredient, calculated as theophylline.

In some related embodiments, the transdermal patch is adapted to deliver a mean active ingredient flux of at least about 5 μg/cm²*h, and in particular of about 10 μg/cm²*h to about 200 μg/cm²*h over a delivery period of at least 24 hours. Again, the amount of active ingredient is calculated as as flux of theophylline. Also in this context, the flux is determined in vitro using pig cadaver skin, optionally using a permeation test model as described herein (see Examples). In further embodiments, the mean active ingredient flux is at least about 15 μg/cm²*h, or on the range from about 15 μg/cm²*h to about 125 μg/cm²*h, respectively.

The performance of the transdermal patch to be selected for carrying out the invention according to some embodiments may also be described in terms of the plasma levels achieved by administering the patch. In some preferred embodiments, the transdermal patch is adapted to provide, during a delivery period of at least 24 hours, mean steady-state plasma concentrations between 0.3 μg/ml and 2.5 μg/ml of theophylline in a human subject. In this context, a delivery period is a designated wearing time for which the transdermal patch is designed, and which is typically provided with the instructions for use of the product.

In some embodiments, the transdermal patch is designed for a wearing time, or delivery period, of about 24 hours. For example, if the patch is prescribed for continuous use over an extended period of time such as several weeks, months or years, the delivery period of 24 hours would mean that a new patch is applied to the skin of the respective user once every day at approximately the same time of the day, at which time the previous patch is also removed. In other preferred embodiments, the transdermal patch is designed for a wearing time, or delivery period, of about 48 hours or two days.

Further preferred are embodiments in which the delivery period is about 3 days or about 72 hours, respectively. In related embodiments, the delivery period is about 3 to 4 day, i.e. the patch is designed to be applied or replaced about twice a week in relatively even intervals. Also related are preferred embodiments in which the delivery period is at least about 4 days, or even about 7 days, respectively. A once weekly dosing interval is considered particularly advantageous for chronic therapies.

In a preferred embodiment, the transdermal patch contains aminophylline as active ingredient and delivers at least about 25 mg aminophylline or 20 mg theophylline per day to an adult human subject for at least 2 consecutive days. This quantity needs to be adapted for children. Again, the transdermal patch is preferably configured to deliver a myelination-specific or remyelination-specific dose of aminophylline to provide a plasma concentration between 0.3 μg/ml and 2.5 μg/ml of theophylline in blood plasma of a human patient.

The transdermal patch may in principle be designed either as a matrix patch or as a reservoir system. A reservoir system is characterised in that it comprises a drug reservoir typically consisting of a liquid or semi-solid non-adhesive formulation containing the active ingredient, such as a liquid solution, covered by permeable membrane that controls the release of the active ingredient into the skin. A matrix patch, on the other hand, comprises the active ingredient in a flexible matrix layer which may also serve as the adhesive layer that ensures the adherence of the patch to the skin. Such design is also referred to as drug-in-adhesive design. Various combinations of features of reservoir systems and matrix patches are also known.

In some of the preferred embodiments, the transdermal patch is designed as a matrix patch comprising a matrix layer, wherein the active ingredient is uniformly distributed in the matrix layer. The inventors have found that matrix patches are indeed capable of incorporating and delivering high amounts of active ingredient as may be necessary for achieving and maintaining plasma concentrations of theophylline that are effective in the prevention or treatment of hypo- or demyelinating diseases and conditions. This advantage is particularly pronounced if aminophylline is used as active ingredient in the patch. Accordingly, in some preferred embodiments, the transdermal patch is designed as a matrix patch comprising a matrix layer in which aminophylline is uniformly distributed.

In some further embodiments, the matrix layer comprises at least about 5 wt. % of the active ingredient. i.e. aminophylline, theophylline or a salt thereof, based on the total weight of the dry matrix layer. Optionally, the matrix layer comprises at least about 10 wt % of the active ingredient, or at least about 20 wt %, such as about 20 wt % to about 45 wt. %. Particularly preferred are embodiments in which the active ingredient is aminophylline, in particular anhydrous aminophylline, and wherein the content of aminophylline is at least about 5 wt %, or at least about 10 wt %, such as from about 10 wt % to about 60 wt %. In other preferred embodiments, the matrix layer comprises at least about 15 wt. % of aminophylline, or at least about 20 wt. %, such as in the range from about 20 wt % to about 45 wt. %, or from about 25 wt % to about 45 wt %, respectively.

The active ingredient may be dissolved or suspended in the matrix layer. While it is assumed that only dissolved active ingredient can be released from the patch and taken up by the skin, it should be kept in mind that a suspension typically also comprises dissolved active ingredient, wherein the amount of the active ingredient in solution reflects its saturation concentration. Suspended particles of the active ingredient allow the drug content of the matrix layer to be further increased, i.e. above saturation, and may function as an active ingredient depot which may be useful for patches designed to be worn over several days. In some preferred embodiments, the matrix layer comprises the active ingredient, in particular aminophylline, which is suspended in the matrix layer.

The matrix layer typically requires one or more matrix polymers or copolymers. In some embodiments, the matrix layer of the transdermal patch disclosed herein comprises a matrix polymer or copolymer comprising an optionally derivatised cellulose ether or cellulose ester, poly[meth]acrylate, polyvinylpyrrolidone, polyvinyl alcohol, polyvinyl acetate, polyisobutylene, polysiloxane, or polyurethane. Examples of potentially useful cellulose derivatives include, without limitation, methylcellulose, ethylcellulose, hydroxypropyl cellulose or hyprolose (HPC), hydroxypropyl methylcellulose or hypromellose (HPMC), hydroxypropyl methylcellulose acetate succinate (HPMC-AS), and sodium carboxymethylcellulose (CMC). HPMC or HPC may be available at different viscosity grades, with a molecular weight (MW) grade ranging from 20 to 1.500 kDa. Examples of such HPMC are HPMC 603, HPMC 645, HPMC 605, HPMC 606 or HPMC 615.

In some preferred embodiments, the matrix layer is a pressure-sensitive adhesive layer, and the matrix polymer is a polymer or copolymer that has itself some pressure-sensitive adhesive properties. Potentially suitable pressure-sensitive adhesive polymers that may be selected for the matrix layer include, without limitation, polymers and copolymers based on acrylates, such as polyacrylates and poly[meth]acrylates; polyisobutylene (PIB), silicone, i.e. polysiloxane, styrene-isoprene-styrene (SIS), and styrene-butadiene-styrene (SBS). Examples of potentially suitable grades of polyacrylates include, acrylate copolymers optionally comprising functional groups such as hydroxyl and/or carboxyl groups, which may be optionally crosslinked and/or further contain vinyl acetate, such as various commercial grades of DURO-TAK and GELVA (by Henkel). Examples of potentially suitable grades of silicone matrix polymers include, without limitation, amine-compatible polymers obtainable by a condensation reaction of a silanol end-blocked polydimethylsiloxane (PDMS) with a silicate resin, followed by capping the residual silanol functionality with trimethylsiloxy groups; such as the commercial grades of Liveo™ BIO-PSA (by DuPont).

In some embodiments, the matrix layer of the transdermal patch comprises at least about 25 wt. % of the matrix polymer, or of the combined matrix polymers in case more than one matrix polymer is used. In the context, the percentage is based on the total weight of the dry matrix layer, including the active ingredient. Also preferred are embodiments in which the amount of the matrix polymer(s) in the matrix layer is in the range from about 25 wt. % to about 80 wt. %, and in particular from about 30 wt. % to about 70 wt. %. If the matrix polymer(s) exhibit(s) pronounced pressure-sensitive adhesive properties as described above, such as pressure-sensitive adhesive silicone, acrylate, polyisobutylene, styrene-isoprene-styrene or styrene-butadiene-styrene matrix polymers, the amount of the matrix polymer(s) may also advantageously be in the range from about 35 wt. % to about 65 wt. %, such as about 35 wt. %, 40 wt. %, 45 wt. %, 50 wt. %, 55 wt. %, or 65 wt. %, respectively. In this context, the expression “about” means±5 wt. %. On the other hand, if the matrix polymer as such is unable to impart sufficient adhesiveness, tack or flexibility to the matrix layer, such that the incorporation of a plasticiser and/or a tackifier is required, as in the case of cellulose derivatives such as hypromellose, the amount of the matrix polymer(s) in the matrix layer may also advantageously be in the range from about 25 wt % to about 65 wt. %, or from about 30 wt % to about 60 wt. %, such as about 30 wt %, 35 wt %, 40 wt %, 45 wt %, 50 wt %, 55 wt %, or 60 wt. %, respectively. In the case of HPMC being selected as matrix polymer, its amount in the matrix layer may also be selected in the range from about 30 wt. % to about 60 wt %, relative to the weight of the dry matrix layer.

In some embodiments, the amounts of the active ingredient and of the matrix polymer or combination of matrix polymers is selected such that the weight ratio of the active ingredient to the matrix polymer(s) is in the range from about 0.5 to about 1.5. In this context, the weight ratio is calculated on the basis of the weight of the dry active ingredient as incorporated in the matrix layer. For matrix layers comprising aminophylline as active ingredient and a pressure-sensitive adhesive polymer as matrix polymer, such as silicone, acrylate, polyisobutylene, styrene-isoprene-styrene or styrene-butadiene-styrene, the weight ratio of the active ingredient to the matrix polymer(s) may also be in the range from about 0.5 to about 1, according to some further preferred embodiments.

In some further preferred embodiments, the matrix layer comprises a permeation enhancer. Permeation enhancers, also known as penetration enhancers or absorption enhancers, are substances that may increase the permeability of the skin and facilitate the absorption of the active ingredient. The stratum corneum, which represents the outermost layer of the skin, acts as a barrier that limits the passage of molecules, including drug substances, from the external environment into the body. Permeation enhancers work by altering the structure and properties of the stratum corneum, making it more permeable to active substances.

Without wishing to be bound by theory, it is believed that there are several potential mechanisms by which permeation enhancers function, including the following mechanisms: disruption of lipid bilayers, increasing skin hydration, opening tight junctions, solubilization of stratum corneum lipids, and/or modulation of protein structure. The disruption of the lipid bilayers of the stratum corneum would reduce the barrier function of the skin. This disruption thereby increases the diffusion of drugs and other molecules through the skin. The hydrating of the stratum corneum can lead to swelling which results in increased permeability. Hydration loosens the tight packing of skin cells and allows molecules to pass through more easily. Tight junctions are specialised intercellular structures that limit the passage of molecules between cells. Permeation enhancers can alter these junctions, creating temporary gaps and facilitating the movement of drugs. In other cases, enhancers are believed to solubilise or dissolve the lipid components of the stratum corneum, disrupting its integrity and allowing for increased drug diffusion. Moreover, some enhancers may interact with skin proteins, altering their structure and function. This can also lead to changes in the barrier properties of the stratum corneum.

The permeation enhancer may, for example, be selected from a monoterpene, a solvent, a lactam, a fatty acid, a fatty acid derivative, an amino acid derivative, α-tocopherol, and/or d-α-tocopheryl polyethylene glycol 1000 succinate.

Monoterpenes belong to the class of terpenes and consist of two isoprene units. In the context of the present invention, acyclic, bicyclic or monocyclic monoterpenes can be used. In addition, also modified terpenes can be used in the context of the present invention. Preferably, the monoterpene is a fatty monoterpene, such as for example aliphatic monoterpenes, in particular myrcene, ocimene. In another embodiment, cyclic monoterpenes can be used, such as, for example, alpha-pinene, camphene, limonene, phellandrene, pinene, sabinene, terpinene. In an alternative embodiment, aromatic monoterpenes can be used, including, but not limited to cymene. In a further alternative embodiment, alcoholic monoterpenes can be used, such as, for example, alcoholic monoterpenes, in particular borneol, carveol, dihydrocarveol, geraniol, menthol, linalool, perilla alcohol, sabinene hydrate, terpineol. In another embodiment, monoterpenes with carbonyl group can be used, including, but not limited to camphor, menthone, carvone, citral, cuminaldehyde, dihydrocarvone, fenchone, safranal, thujone. In an alternative embodiment, phenolic monoterpenes can be used, such as, for example, anethole, carvacrol, eugenol, eucalyptol, methyl cavicol, thymol, trans- anethole, picrocrocin. In yet another preferred embodiment, monoterpenes-related compounds or modified terpenes can be used, including, but not limited to methylbutyryloxy-1-propenylbenzene, allyltetramethoxybenzene, anisaldehyde, anisketone, apiol, elemicine, hydroxyanetholmethylbutyric acid ester, zingerone, phenylpropanes, 5-methoxyl-(2-methylbutyryloxy)-1-propenylbenzene, allyltetramethoxybenzene, anethole, cuminaldehyde, elemicin, estragole, eugenol, eucalyptol, foeniculin, hydroxyanetholmethylbutyric acid ester, methylchavicol, myristicin, safrole, trans-anethole, zingerone. The use of alcoholic monoterpenes is preferred in the context of the present invention. One of the preferred monoterpenes to be used in the present invention is geraniol. It has been surprisingly found by the inventors of the present invention that a constant slow delivery of aminophylline or theophylline is achieved by using a monoterpene as permeation enhancer, in particular in combination with HPMC being used as a matrix polymer.

For example, the inventors have found that a transdermal patch with a matrix layer that incorporates hypromellose (HPMC) as matrix polymer and geraniol as permeation enhancer achieve a remarkable skin flux of the active ingredient according to the invention, in particular of aminophylline. Accordingly, one of the preferred embodiments relates to the selection of hypromellose as the matrix polymer in combination with geraniol as permeation enhancer, and preferably also the incorporation of polyethylene glycol as plasticiser in the matrix layer.

In other embodiments, a solvent is used as permeation enhancer, which solvent may, for example, be selected from diethylene glycol monoethyl ether, octyl dodecanol, oleyl alcohol, dipropylene glycol, propylene glycol, or 1,2-butylene glycol. Other potentially useful solvents that may be employed as permeation enhancers are glycerol, propylene glycol, dimethyl sulfoxide, N-methylpyrrolidone, 2-(2-ethoxyethoxy)ethanol, dimethylformamide, 1-dodecylazacycloheptan-2-one or N-dodecylpyrrolidone. One of the preferred solvents is diethylene glycol monoethyl ether.

In some of the preferred embodiments, the matrix polymer is a silicone adhesive, and the permeation enhancer is diethylene glycol monoethyl ether. The inventors have found that this is a particularly useful combination for achieving a very high flux, in particular of aminophylline. Moreover, patches with matrix layers based on a silicone adhesive, for example an amine-compatible polymers obtainable by a condensation reaction of a silanol endblocked polydimethylsiloxane (PDMS) with a silicate resin, followed by capping the residual silanol functionality with trimethylsiloxy groups, which further comprise diethylene glycol monoethyl ether as permeation enhancer can incorporate relatively large amounts of the active ingredient, in particular aminophylline, without losing their pressure-sensitive adhesive properties or flexibility, in addition to achieving high flux rates of the active ingredient through the skin.

In other embodiments, the permeation enhancer is a fatty acid or fatty acid derivative, such as oleic acid, dodecanol, sodium dodecyl sulphate, potassium dodecyl sulphate, ammonium dodecyl sulphate, sodium octyl sulphate, potassium dodecyl sulphate, ammonium dodecyl sulphate, isopropyl myristate, oleyl oleate, ethyl oleate, glycerol monolaurate, ascorbyl palmitate, sorbitan monolaurate, sorbitan monooleate, sorbitan monopalmitate, sorbitan trioleate, sorbitan monostearate, sorbitan tristearate, polyoxyethylene (20) sorbitan monolaurate, polyoxyethylene (20) sorbitan monopalmitate, polyoxyethylene (20) sorbitan monostearate, isopropyl palmitate, isopropyl myristate, propylene glycol monolaurate, propylene glycol monocaprylate, or any other caprylate, caprate, laurate, linoleate, oleate, palmitate, stearate, isostearate.

The amounts of the permeation enhancer in the matrix layer should be selected in view of the active ingredient, but also in view of the nature of the permeation enhancer itself and of the matrix polymer. In some embodiments, the matrix layer comprises from about 2 wt % to about 20 wt % of the permeation enhancer, or of the combination of permeation enhancers if more than one is used. In other embodiments, the amount of permeation enhancer in the matrix layer is from about 3 wt. % to about 15 wt %, such as about 5±2 wt % or about 10±3 wt %, relative to the total weight of the dry matrix layer.

As mentioned, the matrix layer of the transdermal patch may further comprise a plasticiser, in particular if a matrix polymer is used which is as such not soft, flexible and/or adhesive enough when shaped as a film or layer, such as in the case of cellulose derivatives. Plasticisers are typically low molecular weight resins or liquids which cause a reduction in polymer-polymer chain secondary bonding, forming secondary bonds with the polymer chains instead. Plasticisers may improve film forming properties and the appearance of the film, decreasing the glass transition temperature of the polymer, thereby preventing film cracking, increasing film flexibility and obtaining desirable mechanical properties. In addition, a plasticiser used in the context of certain aspects of the present invention may allow or contribute to a controlled release of aminophylline or theophylline over several days.

In some embodiments, the plasticiser is selected from the group consisting of ethylene glycol, propylene glycol, 1,2-butylene glycol, 2,3-butylene glycol, styrene glycol, polyethylene glycol, glycol ether, propylene glycol monoethyl ether, ethylene glycol monoethyl ether, diethylene glycol monoethyl ether, sorbitol lactate, ethyl lactate, butyl lactate, ethyl glycolate, allyl glycolate, monoethanolamine, diethanolamine, triethanolamine, monisopropanolamine, triethylenetetramine, 2-amino-2-methyl-1,3-propanediol, triethyl citrate, triacetyl glycerol. In some embodiments, the transdermal patch of the present invention comprises phthalate esters, phosphate esters, fatty acid esters and glycol derivatives. In some further preferred embodiments, the plasticiser used in the matrix layer is selected from PEG300, PEG400, PEG600, PEG800, or PEG3350.

Preferably, the plasticiser of the present invention, if present, is used in an amount ranging from about 5 wt % to about 25 wt %, relative to the weight of the dry matrix layer, preferably ranging from about 8 wt % to about 20 wt %, such as from about 10 wt % to about 18 wt %. In some further embodiments, the matrix layer comprises a polymer selected from cellulose derivative such as hypromellose, and a plasticiser selected from polyethylene glycols. The weight ratio of the cellulose derivative to the polyethylene glycol may, for example, be selected in the range from about 1 to 10, and more preferably in the range from about 1.5 to 5, from about 2 to about 3.5, or from about 2.5 to about 4, respectively.

In some further embodiments, the matrix layer comprises a tackifier. Tackifiers, in this context, should be understood as substances that provide or enhance adhesive properties to the skin-facing layer of the patch, typically the matrix layer. In other words, tackifiers help the patch adhere to the skin firmly and maintain its position over an extended period of time. They increase the stickiness or tack of the matrix layer and ensure that it remains in close contact with the skin's surface without coming off easily during normal activities, such as movement, sweating, or contact with clothing. A tackifier may also enhance the ability of the patch to conform to the contours of the skin, which is also relevant for ensuring uniform contact and drug delivery across the application area. Moreover, a tackifiers may contribute to the durability of the patch and to withstand external factors like friction, moisture, and temperature changes without losing its adhesiveness. Furthermore, a tackifier may play a role in controlling the release of the drug from the patch by affecting the rate of drug diffusion through the adhesive layer.

The tackifier may, for example, be a synthetic polymer from the same chemical class as the matrix polymer, such as a polyisobutylene, polyacrylate, or an ethylene-vinyl acetate copolymers, selected within a specific molecular weight range or having a chemical modification that is associated with its ability to increase the tack. Alternatively, certain natural resins and gums may also be used as tackifiers, such as rosin derivatives or terpene-based materials. Also potentially useful as tackifiers are waxy or semi-solid materials, or combinations of two or more materials from the groups above.

With respect to the quantitative composition, it has already been mentioned that the matrix layer preferably comprises at least about 5 wt. % of the active ingredient. i.e. aminophylline, theophylline or a salt thereof. In some further preferred embodiments, the matrix layer preferably comprises from about 20 wt % to about 45 wt. % of the active ingredient; from about 30 wt % to about 70 wt % matrix polymer(s); from about 2 wt % to about 20 wt % permeation enhancer(s); and optionally a plasticiser and/or tackifier.

Also preferred are embodiments in which the matrix layer comprises from about 20 to about 45 wt % of the active ingredient; from about 35 wt % to about 65 wt % of one or more matrix polymers selected from pressure-sensitive adhesive silicone, acrylate, polyisobutylene, styrene-isoprene-styrene or styrene-butadiene-styrene polymers; from about 3 wt % to about 15 wt % permeation enhancer(s); and optionally a plasticiser and/or tackifier. Also in this context, aminophylline is a preferred active ingredient.

In alternative preferred embodiments, the matrix layer comprises from about 20 to about 45 wt % of the active ingredient; from about 30 wt % to about 60 wt % of one or more matrix polymers selected from a cellulose ether such as hypromellose, from about 3 wt % to about 15 wt % permeation enhancer(s), from about 5 wt % to about 25 wt % plasticiser(s), and optionally a tackifier. Again, aminophylline is a preferred active ingredient also in the context of these embodiments.

Another preferred transdermal patch comprises aminophylline or, alternatively, theophylline and HPMC as matrix polymer, and/or PEG as plasticizer and/or geraniol as permeation enhancer. If the transdermal patch of the present invention is composed of aminophylline, HPMC, PEG and a monoterpene such as geraniol, HPMC is present in an amount ranging from 30% to 65% by dry weight in the matrix layer, said PEG is present in an amount ranging from 5% to 25% by dry weight in the matrix layer, and said monoterpene or geraniol is present in an amount ranging from 3% to 20% by dry weight in the matrix layer.

If appropriate, other components may be added to the transdermal patch formulation of the present intervention such as, but not limited to, preservatives and antioxidants. Said preservatives may be selected from, for example, ethyl p-hydroxy benzoate, propyl p-hydroxybenzoate, butyl p-hydroxy benzoate. Said antioxidants may be selected from, for example, tocopherol and its ester derivates, ascorbic acid, ascorbic acid-stearic acid ester, dibutyl hydroxy toluene (BHT), butyl hydroxy anisole (BHA) or sodium metasulphite. These components are preferably incorporated in the matrix layer, if present. The transdermal patch may further comprise a removable protective release liner, which protects the transdermal patch during storage and is removed before application to the skin.

When attached, the release liner covers the side or layer of the patch that is intended to be applied to the skin. The removable protective release liner may preferably comprise one or more of polyester, polyvinyl chloride, polypropylene, polyamide, polyvinylchloride, aluminium, or polyethylene glycol terephthalate film. In preferred embodiments, the removable protective release liner is impermeable to the drug and easily detachable from the patch. For ensuring its removability, the release liner may be coated or pretreated with one or more of silicone, fluorosilicone, fluorocarbon and polyethylene if required. Specific examples of potentially suitable release liner materials include, without limitation, foils of the PRIMELINER™ series (Loparex), and fluoro-coated polyester release liners supplied by 3M, such as Scotchpak™ fluoropolymer-coated polyester release liners.

Moreover, in this context of release liners it should be understood that the expression “transdermal patch” may be used to describe the respective product with or without its release liner, i.e. whether before or after its removal from the release liner.

In some preferred embodiments, the transdermal patch further comprises a backing layer. The backing layer is the outermost layer of the patch, it forms the opposite side of the patch which does not adhere to the skin. It provides a protective barrier between the patch's contents and the external environment and thus prevents contamination, evaporation, and leakage of the ingredients contained within the patch. The backing layer is typically made from materials that are durable and resistant to wear and tear. This helps maintain the integrity of the patch over its intended wear time. The backing layer is substantially impermeable to the active ingredient. Moreover, the backing layer may be occlusive or non-occlusive. As used herein, the expression “occlusive” means that the backing layer is substantially impermeable to water and water-vapor.

Potentially suitable materials for a backing layer are generally well-known in the art, and include, in particular, polyethylene terephthalate (PET), polyethylene (PE), ethylene vinyl acetate-copolymer (EVA), polyurethanes, polyvinyl chloride, and mixtures thereof. Other potentially useful materials include, without limitation, polyethylene, polypropylene, polybutadiene, vinyl acetate-vinyl chloride copolymers, polyvinyl chloride, polyvinyl acetate, polyvinylidene chloride, polyethylene, polyimide, polyester, polyurethane, as well as a laminates of two or more of the above, or laminates of any of these with paper or aluminium.

Suitable backing layers thus represent, for example, PET laminates, EVA-PET laminates and PE-PET laminates. Also suitable are woven or non-woven backing materials, or foamed polymer films. In some cases, the backing layer may itself be a laminate of several sub-layers.

A typical backing material has a thickness in the range of about 2 μm to about 1000 μm, in particular of about 10 μm to about 300 μm. Suitable backing materials include commercially available backings films, such as breathable backings such as CoTran™ (3M), backings which exhibit low moisture vapor transmission rate and high oxygen transmission, and non-breathable polyester-based laminate backings such as Scotchpak® (3M), such as Scotchpak 9732 (3M). In some specific embodiments, the backing layer is a stretchable, occlusive backing layer comprising a stretchable fabric coated with an occlusive polymer coating comprising a styrene-isoprene-styrene block copolymer and tackifier, for example as described in WO 2017/087354.

In some further embodiments, a transdermal patch is provided which comprises (a) a backing layer; (b) a matrix layer comprising aminophylline, a matrix polymer, a plasticizer, and a permeation enhancer; (c) a removable protective release liner; for use in the treatment or prevention of a hypomyelinating or a demyelinating disease or condition or a lesion of the peripheral or central nervous system where demyelination occurs. Further optional features and preferences to the patch components as described herein-above are also applicable to these embodiments.

In another aspect, the present invention relates to the use of aminophylline or theophylline or a salt thereof for the manufacture of a medicament for treating or preventing a hypomyelinating or demyelinating disease or condition, wherein the medicament comprises a pharmaceutical composition in the form of a transdermal patch, and wherein the aminophylline or theophylline or salt thereof is the sole active ingredient in the transdermal patch.

In a related aspect, the invention relates to a method of treating a subject affected with, or at risk of becoming affected with, a hypomyelinating or demyelinating disease or condition, the method comprising a step of administering a pharmaceutical composition comprising an active ingredient selected from aminophylline or theophylline or a salt thereof, wherein the composition is in the form of a transdermal patch, and wherein the aminophylline or theophylline or salt thereof is the sole active ingredient in the transdermal patch.

The transdermal patches described herein can be prepared by methods generally known in the art. As one step, the matrix layers described herein can be prepared by methods known in the art, such as blending or mixing the matrix polymer(s) in powder or liquid form with an appropriate amount of active ingredient in the presence of an appropriate solvent, such as a volatile organic solvent and/or water, optionally with other excipients, in particular the permeation enhancer. To form a final product, the mixture comprising the components above may be cast or coated onto a release liner (optionally, at ambient temperature and pressure) followed by evaporation of the solvent(s), for example, at room temperature, or slightly elevated temperature, or by a heating or drying step, to form the drug-containing polymer matrix on a release liner. A backing layer may be applied to form a laminate from which individual patches may be cut or punched out.

As mentioned, the transdermal patch according to any embodiment disclosed herein can be used for any demyelinating disease or demyelinating lesion, because it promotes or accelerates myelination or remyelination of affected nerves of a patient. Demyelinating diseases result in a loss of myelin, which can cause neurological defects, such as vision changes, weakness, altered sensation and behavioural or cognitive problems. The symptoms of demyelination correspond to the affected area of the nervous system. Demyelination affecting the lower spine or the spinal nerves causes sensory changes or weakness of the legs. It may also be associated with a bowel and bladder control. Demyelination in the brain can cause a variety of problems, such as impaired memory or decreased vision. The transdermal patch can be used for the treatment or prevention of any hypomyelinating or demyelinating disease or condition. Preferably, said hypomyelinating or demyelinating disease or condition is selected from the group consisting of leukodystrophy, demyelinating diseases of the central nervous system, multiple sclerosis, central pontine myelinolysis, glioma, schizophrenia, demyelination due to aging, diabetes or due to toxic agents, chronic inflammatory demyelinating polyneuropathy (CIDP), Charcot- Marie-Tooth disease (CMT), Guillain-Barré syndrome, hereditary neuropathy with liability to pressure palsy (HNPP), progressive inflammatory neuropathy, Dejerine-Sottas disease, Waardenburg syndrome, congenital hypomyelinating neuropathy (CHN), Cowchock syndrome, Rosenberg-Chutorian syndrome, or Roussy-Levy syndrome, demyelination after traumatic lesion of the PNS or CNS. The use of the transdermal patch for the treatment of a demyelinating disease affecting the peripheral nervous system, including, but not limited to Guillain-Barré syndrome or Charcot-Marie-Tooth (CMT) disease, or the treatment of a demyelinating disease affecting the central nervous system, including, but not limited to multiple sclerosis, is preferred.

The transdermal patch of the present invention can also be used in the treatment of a demyelinating lesion, including, but not limited to traumatic lesion of the nervous system, peripheral nerve injury or spinal cord injury.

Further embodiments disclosed herein include, without limitation, the following items:

(1) A transdermal patch comprising:
a. a backing layer
b. a matrix layer comprising aminophylline or theophylline or a salt thereof, a matrix polymer, a plasticizer, a permeation enhancer
c. a removable protective release liner for use in the treatment or prevention of a hypomyelinating or a demyelinating disease or condition or a lesion of the peripheral or central nervous system where demyelination occurs.
   (2) The transdermal patch for the use according to item 1, wherein said patch comprises an additional skin adhesive layer.
   (3) The transdermal patch for the use according to item 1 and 2, wherein said patch is configured to release aminophylline, theophylline or a salt thereof over a period of up to 4 consecutive days, or longer, through the skin of a human or animal subject.
   (4) The transdermal patch for the use according to any one of items 1 to 3, wherein said patch is configured to deliver a myelination-specific or remyelination-specific dose of aminophylline to provide a plasmatic concentration between 0.3 μg/ml and 2.5 μg/ml of theophylline in blood plasma of a human or animal subject.
   (5) The transdermal patch for the use according to any one of items 1 to 4, wherein said matrix polymer is selected from the group consisting of a cellulose polymer (hydroxypropyl methylcellulose (HPMC), hydroxypropyl methylcellulose acetate succinate (HPMC-AS), ethyl cellulose (EC), hydroxypropyl cellulose (HPC), microcrystalline cellulose (MCC)), a polymer or copolymer of acrylic acid, methacrylic acid, an acrylic acid ester and/or methacrylic acid ester residues, a polymer or copolymer of vinylpyyrolidone, vinyl alcohol and/or vinyl acetate, polyisobutylenes, polysiloxanes, polyurethanes, and silicon polymers.
   (6) The transdermal patch for the use according to any one of items 1 to 5, wherein said plasticizer is selected from the group consisting of ethylene glycol, propylene glycol, 1,2-butylene glycol, 2,3-butylene glycol, styrene glycol, polyethylene glycol, glycol ether, propylene glycol monoethyl ether, ethylene glycol monoethyl ether, diethylene glycol monoethyl ether, sorbitol lactate, ethyl lactate, butyl lactate, ethyl glycolate, allyl glycolate, monoethanolamine, diethanolamine, triethanolamine, monoisopropanolamine, triethylenetetramine, 2-amino-2-methyl-1,3-propanediol, triethyl citrate, triacetyl glycerol.
   (7) The transdermal patch for the use according to any one of items 1 to 6, wherein said permeation enhancer is a monoterpene, a solvent, a lactam, a fatty acid, a fatty acid derivative, an amino acid derivative, α-tocopherol, and/or d-α-tocopheryl polyethylene glycol 1000 succinate.
   (8) The transdermal patch for the use according to item 7, wherein said monoterpene is selected from the group consisting of
a. aliphatic monoterpenes, in particular myrcene, ocimene;
b. cyclic monoterpenes, in particular alpha-pinene, camphene, limonene, phellandrene, pinene, sabinene, terpinene;
c. aromatic monoterpenes, in particular cymene;
d. alcoholic monoterpenes, in particular borneol, carveol, dihydrocarveol, geraniol, menthol, linalool, perilla alcohol, sabinene hydrate, terpineol;
e. monoterpenes with carbonyl group, in particular camphor, menthone, carvone, citral, cuminaldehyde, dihydrocarvone, fenchone, safranal, thujone;
f. phenolic monoterpenes, in particular anethole, carvacrol, eugenol, eucalyptol, methyl cavicol, thymol, trans-anethole, picrocrocin;
g. monoterpenes-related compounds, in particular methylbutyryloxy-1-propenylbenzene, allyltetramethoxybenzene, anisaldehyde, anisketone, apiol, elemicine, hydroxyanetholmethylbutyric acid ester, zingerone, phenylpropanes, 5-Methoxyl-(2-Methylbutyryloxy)-1-propenylbenzene, allyltetramethoxybenzene, anethole, cuminaldehyde, elemicin, estragole, eugenol, eucalyptol, foeniculin, hydroxyanetholmethylbutyric acid ester, methylchavicol, myristicin, safrole, trans-anethole, Zingeron.
   (9) The transdermal patch for the use according to item 7, wherein said fatty acid or fatty acid derivative is oleic acid, dodecanol, sodium dodecyl sulphate, potassium dodecyl sulphtae, ammonium dodecyl sulphate, sodium octyl sulphate, potassium dodecyl sulphate, ammonium dodecyl sulphate isopropyl myristate, oleyl oleate, ethyl oleate, glycerol monolaurate, ascorbyl palmitate, sorbitan monolaurate, sorbitan monooleate, sorbitan monopalmitate, sorbitan trioleate, sorbitan monostearate, sorbitan tristearate, polyoxyethylene (20) sorbitan monolaurate, polyoxyethylene (20) sorbitan monopalmitate, polyoxyethylene (20) sorbitan monostearate.
   (10) The transdermal patch for the use according to item 7, wherein said amino acid derivative is dodecyl 2-dimethylaminoproprionate, dodecyl N-acetylproline, N-laurylsarcosine, dodecyl 6-dimethylhexanoate.
   (11) The transdermal patch for the use according to any one of items 1 to 10, wherein said matrix polymer is HPMC, and/or said plasticizer is PEG and/or said permeation enhancer is Geraniol.
   (12) The transdermal patch for the use according to any one of items 1 to 11, wherein said hypomyelinating or demyelinating disease or condition is selected from the group consisting of leukodystrophies, demyelinating diseases of the central nervous system, multiple sclerosis, central pontine myelinolysis, glioma, schizophrenia, demyelination due to aging, diabetes or due to toxic agents, chronic inflammatory demyelinating polyneuropathy (CIDP), Charcot-Marie-Tooth disease (CMT), Guillain-Barré syndrome, hereditary neuropathy with liability to pressure palsy (HNPP), progressive inflammatory neuropathy, Dejerine-Sottas disease, Waardenburg syndrome, congenital hypomyelinating neuropathy (CHN), Cowchock syndrome, Rosenberg-Chutorian syndrome, or Roussy-Levy syndrome.
   (13) The transdermal patch for the use according to any one of items 1 to 12, wherein said injury is selected from the group consisting of traumatic lesion of the nervous system, peripheral nerve injury or spinal cord injury.
   (14) A pharmaceutical composition, comprising aminophylline or theophylline, a matrix polymer, a plasticizer and a permeation enhancer for use as a medicament for promoting or accelerating myelination and/or remyelination used in the treatment or prevention of a hypomyelinating or a demyelinating disease or condition or a lesion of the peripheral or central nervous system where demyelination occurs.
   (15) The pharmaceutical composition for the use according to item 14, wherein said matrix polymer is HPMC, and/or said plasticizer is PEG and/or said permeation enhancer is Geraniol.

### DETAILED DESCRIPTION OF THE DRAWINGS

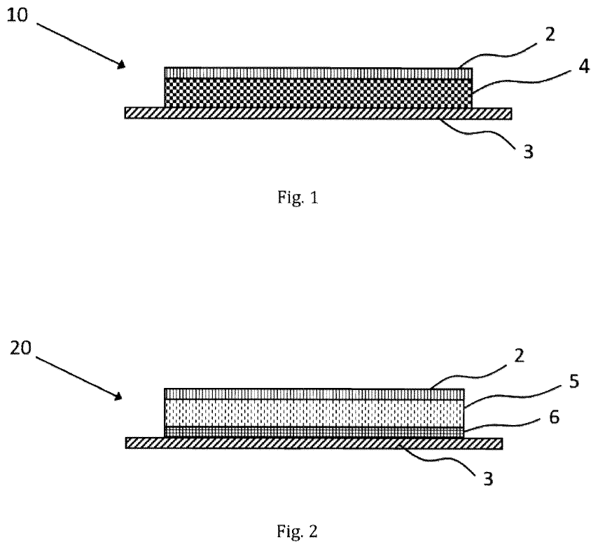

FIGS. 1 to 4, none of which are drawn to scale, exemplify simplified, schematic representations of basic transdermal patch designs that may be used to carry out the invention in some of its aspects.

FIG. 1 show a cross-sectional view of an example of a transdermal patch (10) according to an embodiment, comprising a backing layer (2), a release liner (3), and a self-adhesive matrix layer (4) which comprises, as sole active ingredient, aminophylline or theophylline or salt thereof. The active ingredient is uniformly or homogeneously distributed in the matrix layer (4), whether dissolved or also suspended therein. The matrix layer (4) exhibits adhesive properties (self-adhesive or pressure-sensitive adhesive) since in this design no other layer is available for ensuring adhesion to the skin. Such patch design may also be referred to as a matrix system or matrix patch, or a transdermal patch with a drug-in-adhesive design.

In the exemplary embodiment, the inert backing layer (2) which protects the matrix layer (4) during storage, handling and use of the transdermal patch (10) has, at least in the depicted cross-section, the same length (and/or width) as the matrix layer (4). In contrast, the size (length and/or width) of the release liner (3) is larger than that of the other layers (2, 4), which is convenient for the user as it facilitates the removal of the part of the patch which is applied to the skin, i.e. the matrix layer (4) together with the backing layer (2).

No top view of the patch (10) if FIG. 1 is shown. It should be mentioned, however, that the overall shape of the patch (10) as would be depicted by a top view could be that of a circular or oval disk, or a square or rectangle.

FIG. 2 show a cross-sectional view of an example of a transdermal patch (20) according to a further embodiment. As in the embodiment shown in FIG. 1, the patch (20) comprises a backing layer (2) and a release liner (3). In this case, however, the active ingredient is contained in a reservoir (5), such as a gel composition surrounded by a semi-permeable membrane which controls the rate of release of the active ingredient from the reservoir (5). An adhesive layer (6) is arranged between the reservoir (5) and the release liner (3) to ensure skin adhesion of the patch when in use. Such transdermal patch design may also be referred to as a reservoir system.

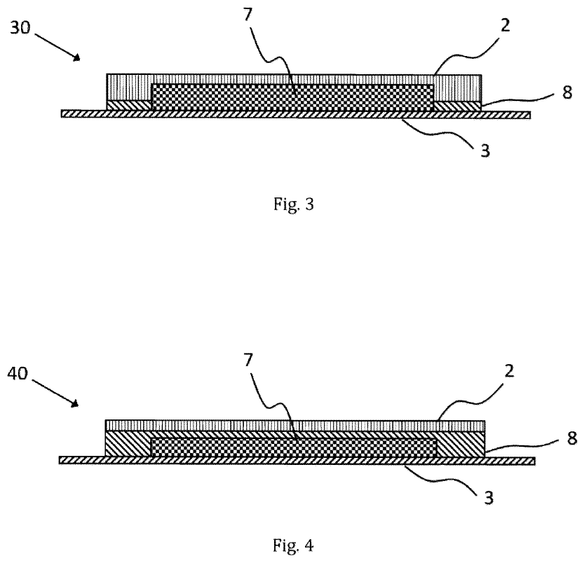

FIG. 3 depicts a cross-sectional view of an example of a transdermal patch (30) according to a yet further embodiment. The patch (30) is, in this case, may also be a matrix system in that the active ingredient, i.e. aminophylline or theophylline or salt thereof, may be uniformly or homogeneously distributed in a matrix layer (7) which is arranged between a backing layer (2) and a release liner (3). The patch design differs from that shown in FIG. 1 in that the size (i.e. area) of the backing layer (2) is larger than that of the matrix layer (4). Where the backing layer (2) extends beyond the matrix layer (7), an auxiliary adhesive layer (8) may be arranged as the backing layer (2) itself does not normally have self-adhesive properties. The auxiliary adhesive layer (8) may, for example, have an annular shape if the matrix layer (7) represents a circular disk when seen from the top (not shown). Such design may be useful in case the matrix layer (7) has no or insufficient self-adhesiveness such that the adhesion of the transdermal patch (30) to the skin needs to be supported by the auxiliary adhesive layer (8).

FIG. 4 depicts a cross-sectional view of an example of a transdermal patch (40) according to a yet further embodiment. The design of the patch (40) is similar to that shown in FIG. 3 in that it also has a release liner (3), a backing layer (2), and a matrix layer (7) whose size (i.e. in terms of area) is smaller than that of the backing layer (2). In contrast to the patch (30) of FIG. 3, however, the auxiliary adhesive layer (8) extends over the same area as the backing layer (2), entirely covering the matrix layer (7). In principle, such patch design may serve the same purpose as that of FIG. 3, i.e. to enhance the skin adhesion of the transdermal patch (40) in case the matrix layer (7) itself has no or insufficient self-adhesiveness. Potentially, however, the patch (40) may be easier to manufacture than the patch (30) of FIG. 3.

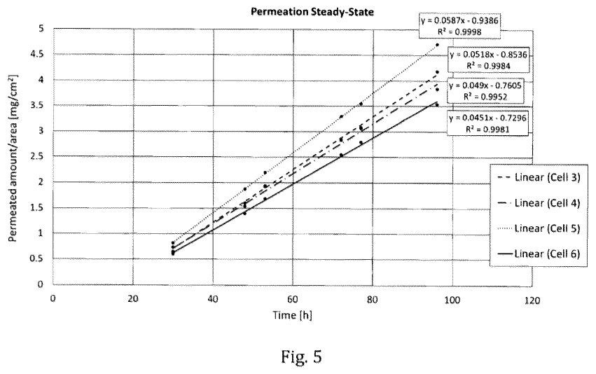

FIG. 5 depicts skin permeation data obtained in Example 1, as described below.

### EXAMPLES

### Example 1

### Preparation of Transdermal Patch

21.875 g of HPMC 606 16% solution was weighed in an amber glass container with stirring stick. 1.05 g of PEG 300 was added to the HPMC solution and mixed until a homogeneous solution was obtained. 2.15 g aminophylline was added and allowed to mix for approx. 2 hours. 700 mg Geraniol was added and let mix for another 2 h for a homogenous distribution. The suspension was poured into a Petri dish and placed in a drying oven at 32° C. until the water had evaporated. The patch was removed from the drying oven when it could be easily detached from the Petri dish without leaving residue in the bottom. The patch was detached and stored in the closed Petri dish until use.

An embodiment of the transdermal patch of the present invention has the composition as provided in Table 1.

TABLE 1

| | | Proportion in | Proportion in | |
|---|---|---|---|---|
| | Quantity | wet mass | final product | |
| Substance | [g] | [wt. %] | [wt. %] | Function |
| Aminophylline | 2.15 | 8.34 | 29.05 | Active |
| | | | | ingredient |
| HPMC 606 (16 | 21.875 | 84.87 | 47.30 | Matrix polymer |
| wt. % aqueous | | | | |
| solution) | | | | |
| PEG 300 | 1.05 | 4.07 | 14.19 | Plasticiser |
| Geraniol | 0.7 | 2.72 | 9.46 | Permeation |
| | | | | enhancer |

### In Vitro Skin Permeation Testing

The skin permeation data given in FIG. 5 was obtained using the following test method with a diffusion cell apparatus (Model: EDC-07, Electrolab India PVT, LTD.). The lower part of the vertical diffusion cells was filled with PBS pH 7.4 as acceptor medium. Across the orifice of the lower part of the diffusion cell, pig cadaver skin was placed so that the acceptor medium contacted the skin. The temperature of the medium was then kept at 32±2° C. A 2.01 cm² patch was punched out of the patch material prepared as described above and placed on top of the pig cadaver skin. The sampling port was covered except when in use. The cells were maintained at 32±2° C. throughout the course of the experiment. The acceptor fluid was stirred by means of a magnetic stirrer throughout the experiment to assure a uniform sample and reduced diffusion barrier on the dermal side of the skin. Samples were taken after 3, 6, 24, 30, 48, 53, 72, 77 and 96 hours and replaced with fresh acceptor medium. The samples were analysed for theophylline concentration using high performance liquid chromatography with an aminophylline calibration curve and theobromine as internal standard (Column: 250×4.0 mm, 7 μm particle size, Mobile phase: 7:93 acetonitrile: 1.36 g/L solution of sodium acetate containing 0.50 percent V/V of glacial acetic acid, Flow rate: 2.0 ml/min, Detector: UV at 272 nm, Injection volume: 20 μL, Run time: 10 minutes) The cumulative amount of active ingredient calculated as theophylline penetrating through the skin was calculated and reported as μg/cm². From this data, the achievable steady state plasma concentration was calculated assuming a patch area of 60 cm² and an average theophylline clearance of 3 L/h. FIG. 5 shows data of cells 3 to 6. The data demonstrate that aminophylline is constantly delivered over the skin, resulting in a steady state plasma concentration of >0.90 μg/ml, and an average flux of 51.15 μg/cm²* hour.

The data further demonstrate that the formulation is suitable for myelination and/or remyelination due to a constant release of the optimal dose in the plasma. By contrast to known transdermal patches, the formulation of the present invention needs not to be administered every day, but steady plasmatic concentration of theophylline can be maintained for at least 4 consecutive days or longer. Known transdermal patches need to be applied every day, whereas the inventive transdermal patch needs to be applied every week or every two weeks only. As such, the transdermal patch of the present invention is suitable for any treatment of a demyelinating disease or lesion involving the promotion or acceleration of myelination and/or remyelination, such as after a traumatic lesion of the nervous system, peripheral nerve injury or a spinal cord injury.

### Example 2

Further formulations useful as matrix layers in transdermal patches were prepared and tested. For each formulation, the wet mass comprising the active ingredient, matrix polymer, permeation enhancer, plasticiser (if present), and water or solvent were combined and stirred until homogenous. As matrix polymers, hypromellose (HPMC 606) and a silicone polymer with pressure-sensitive adhesive properties (Liveo™ BIO-PSA, an amine-compatible silicone polymer obtainable by a condensation reaction of a silanol end-blocked polydimethylsiloxane with a silicate resin, followed by capping the residual silanol functionality with trimethylsiloxy groups), referred to herein below as silicone PSA, were used. Either aminophylline or theophylline were used as active ingredient (API). As permeation enhancers, geraniol and diethylene glycol monoethyl ether, also referred to as 2-(2-ethoxyethoxy)ethanol, were tested. PEG 300 was used as plasticiser in formulations based on hypromellose as matrix polymer.

For the formulations based on HPMC as matrix polymer, a viscous aqueous solution of the polymer was prepared of which the required amount was filled into a mixing vessel and stirred for at least two hours. Next, the enhancer was added and stirring was continued for at least another two hours. Subsequently, the active ingredient was added as a powder. The mass was stirred for at least 12 hours, resulting in a homogeneous, viscous suspension without visible air bubbles. For the silicone-based formulations, the matrix polymer was already provided as a viscous organic solution to which first the enhancer was added, followed by stirring over at least 2 hours, and subsequently the active ingredient, again followed by stirring over at least 12 hours.

The preparation of the matrix layers was performed by coating a transparent, fluoropolymer-coated polyester foil with a thin layer of the respective wet mass by manual blade coating, using a electromotive film applicator (Coatmaster 510 by Erichsen) with a heatable vacuum suction plate for holding the polyester foil and an film applicator similar to a doctor blade having a gap width of 1.0 mm or 2.0 mm, respectively, followed by drying. In one case, a thicker wet film was prepared by two consecutive coating steps using the blade with the 2.0 mm gap. The coating temperature of the suction plate was set at 55° C., and the coating speed was 3.0 mm/s. The films were dried for two hours at 55° C., followed by further drying at room temperature for another 24 hours.

The compositions of the dry matrix layer formulations prepared in this series are provided in Table 2. All amounts are in wt %, relative to the total weight of the dry matrix layer. As can be readily obtained from the amounts, the weight ratio of the active ingredient to the matrix polymer in the formulation samples was about 0.74 except for sample D, where said ratio was about 0.62. It can also be seen that the weight ratio of the matrix polymer to the plasticiser in those formulations that comprises hypromellose in combination with PEG 300 was about 3.33.

TABLE 2

| | Patch sample no. | | | | | | |
|---|---|---|---|---|---|---|---|
| | A | B | C | D | E | F | G |
| | Wet film thickness | | | | | | |
| | 1 mm | 2 mm | >2 mm* | 2 mm | 2 mm | 1 mm | 1 mm |
| Aminophylline | 32.99 | 32.99 | 32.99 | | 32.98 | 38.12 | 38.12 |
| Theophylline | | | | 29.11 | | | |
| Hypromellose | 44.67 | 44.67 | 44.67 | 47.26 | 44.66 | | |
| Silicone PSA | | | | | | 51.57 | 51.57 |
| Geraniol | 8.93 | 8.93 | 8.93 | 9.45 | | | |
| Diethylene glycol | | | | | 8.93 | 10.31 | 10.31 |
| monoethyl ether | | | | | | | |
| PEG 300 | 13.40 | 13.40 | 13.40 | 14.18 | 13.43 | | |

*After drying a wet film of 2 mm thickness, another wet film was coated thereon with the same doctor blade having a gap width of 2 mm; the thickness of the second wet film was therefore 2 mm minus the thickness of the dry first layer.

The samples were subsequently tested their performance with respect to the skin permeation of the active ingredient that they were able to achieve. Essentially the same method as described in Example 1 was used, except that the time periods over which samples of the acceptor medium were withdrawn were up to about 75 hours. Mean active ingredient flux rates were calculated as mean amounts of theophylline that permeated into the acceptor medium per cm² and hour, over the linear portions of the respective testing period, regardless which active ingredient was used. In detail, the flux was calculated by plotting cumulative permeated amounts of active ingredient for each cell of the experiment against time. Linear regression was then performed for the linear portion. For all patch samples and testing periods, it was observed that the permeated amounts over time followed zero-order kinetics, at least until a depletion effect was observed. The mean flux values and their standard deviations (SD) are provided in Table 3. Also provided in the table are the calculated theophylline steady-state plasma concentrations (cₛₛ [μg/ml]) and their standard deviations (SD) which would correspond to these flux values assuming a patch size of 60 cm² and a theophylline plasma clearance of 3 L/h.

TABLE 3

| Patch sample no. | A | B | C | D | E | F | G |
|---|---|---|---|---|---|---|---|
| Flux [μg/cm²*h] | 33.3 | 73.7 | 68.2 | 8.1 | 5.5 | 178.8 | 46.1 |
| SD of flux | 13.3 | 43.5 | 32.9 | 4.7 | 1.9 | 81.3 | 11.7 |
| [μg/cm²*h] | | | | | | | |
| cₛₛ [μg/ml] | 0.67 | 1.47 | 1.36 | 0.16 | 0.11 | 3.58 | 0.92 |
| SD of cₛₛ [μg/ml] | 0.27 | 0.87 | 0.66 | 0.09 | 0.04 | 1.63 | 0.23 |

The results indicate, firstly, that high theophylline flux rates are achievable over long periods of time such as over 60 hours or more, using matrix layer formulations based on cellulose ethers such as hypromellose, or on pressure-sensitive adhesive silicones. Desired active ingredient delivery rates can be tailored for each patch formulation by adjusting the patch size, or the area of the matrix layer which is in contact with the skin. For example, if the target plasma concentrations of theophylline are in range of about 0.3 and 2.5 μg/ml, all formulations are in principle suitable; sample F, which shows a particularly high flux, would be used as a smaller patch having an area substantially smaller than 60 cm², whereas samples D and E may have to be used as larger patches. It is also noted that geraniol is a highly effective permeation enhancer for matrix formulations based on cellulose ethers such as hypromellose, whereas diethylene glycol monoethyl ether is particularly effective as permeation enhancer in combination with a silicone-based matrix polymer. Moreover, the results indicate that aminophylline seems superior to theophylline in terms of skin permeation, which was unexpected as the molar mass of aminophylline (420.4 g/mol) is substantially higher than that of theophylline (180.2 g/mol), in view of the common assumption that larger molecular weights are generally associated with lower permeation rates.

1. A pharmaceutical composition comprising an active ingredient selected from aminophylline or theophylline or a salt thereof for use in the treatment or prevention of a hypomyelinating or demyelinating disease or condition,
wherein the composition is in the form of a transdermal patch, and
wherein the aminophylline or theophylline or salt thereof is the sole active ingredient in the transdermal patch.

2. The pharmaceutical composition according to claim 1, wherein the hypomyelinating or demyelinating disease or condition is selected from leukodystrophies, demyelinating diseases of the central nervous system, multiple sclerosis, central pontine myelinolysis, glioma, schizophrenia, demyelination due to aging, diabetes or due to toxic agents, chronic inflammatory demyelinating polyneuropathy (CIDP), Charcot-Marie-Tooth disease (CMT), Guillain-Barré syndrome, hereditary neuropathy with liability to pressure palsy (HNPP), progressive inflammatory neuropathy, Dejerine-Sottas disease, Waardenburg syndrome, congenital hypomyelinating neuropathy (CHN), Cowchock syndrome, Rosenberg-Chutorian syndrome, Roussy-Levy syndrome, lesion of the peripheral or central nervous system associated with demyelination, traumatic lesion of the nervous system, peripheral nerve injury or spinal cord injury.

3. The pharmaceutical composition according to claim 1, wherein the active ingredient is aminophylline.

4. The pharmaceutical composition according to claim 1, wherein the transdermal patch is adapted to transdermally deliver a dose of at least about 20 mg of the active ingredient to a human subject within 24 hours, wherein the delivered dose is calculated as theophylline.

5. The pharmaceutical composition according to claim 1, wherein the transdermal patch is adapted to deliver a mean active ingredient flux of 10 to 200 μg/cm²*h over a delivery period of at least 24 hours, wherein the flux is calculated as flux of theophylline and determined in vitro using pig cadaver skin.

6. The pharmaceutical composition according to claim 1, wherein the transdermal patch is adapted to provide during a delivery period of at least 24 hours mean steady-state plasma concentrations between 0.3 μg/ml and 2.5 μg/ml of theophylline in a human subject.

7. The pharmaceutical composition according to claim 1, wherein the delivery period is at least 72 hours.

8. The pharmaceutical composition according to claim 7, wherein the delivery period is at least 4 days.

9. The pharmaceutical composition according to claim 1, wherein the transdermal patch is designed as a matrix patch comprising a matrix layer, wherein the active ingredient is uniformly distributed in the matrix layer.

10. The pharmaceutical composition according to claim 9, wherein the matrix layer comprises at least 5 wt. % of aminophylline.

11. The pharmaceutical composition according to claim 9, wherein the active ingredient is suspended in the matrix layer.

12. The pharmaceutical composition according to claim 9, wherein the matrix layer comprises a matrix polymer or copolymer comprising an optionally derivatised cellulose ether, cellulose ester, poly[meth]acrylate, polyvinylpyrrolidone, polyvinyl alcohol, polyvinyl acetate, polyisobutylene, polysiloxane, or polyurethane.

13. The pharmaceutical composition according to claim 9, wherein the matrix layer is a pressure-sensitive adhesive layer.

14. The pharmaceutical composition according to claim 9, wherein the matrix layer comprises a permeation enhancer.

15. The pharmaceutical composition according to claim 14, wherein the permeation enhancer is a monoterpene, a solvent, a lactam, a fatty acid, a fatty acid derivative, an amino acid derivative, α-tocopherol, and/or d-α-tocopheryl polyethylene glycol 1000 succinate.

16. The pharmaceutical composition ter-se according to claim 15, wherein the monoterpene is selected from the group consisting of
a. aliphatic monoterpenes, in particular myrcene, ocimene;
b. cyclic monoterpenes, in particular alpha-pinene, camphene, limonene, phellandrene, pinene, sabinene, terpinene;
c. aromatic monoterpenes, in particular cymene;
d. alcoholic monoterpenes, in particular borneol, carveol, dihydrocarveol, geraniol, menthol, linalool, perilla alcohol, sabinene hydrate, terpineol;
e. monoterpenes with carbonyl group, in particular camphor, menthone, carvone, citral, cuminaldehyde, dihydrocarvone, fenchone, safranal, thujone;
f. phenolic monoterpenes, in particular anethole, carvacrol, eugenol, eucalyptol, methyl cavicol, thymol, trans-anethole, picrocrocin;
g. monoterpene-related compounds, in particular methylbutyryloxy-1-propenylbenzene, allyltetramethoxybenzene, anisaldehyde, anisketone, apiol, elemicine, hydroxyanetholmethylbutyric acid ester, zingerone, phenylpropanes, 5-methoxyl-(2-methylbutyryloxy)-1-propenylbenzene, allyltetramethoxybenzene, anethole, cuminaldehyde, elemicin, estragole, eugenol, eucalyptol, foeniculin, hydroxyanetholmethylbutyric acid ester, methylchavicol, myristicin, safrole, trans-anethole, zingerone.

17. The pharmaceutical composition according to claim 15, wherein the fatty acid or fatty acid derivative is oleic acid, dodecanol, sodium dodecyl sulphate, potassium dodecyl sulphtae, ammonium dodecyl sulphate, sodium octyl sulphate, potassium dodecyl sulphate, ammonium dodecyl sulphate, isopropyl myristate, oleyl oleate, ethyl oleate, glycerol monolaurate, ascorbyl palmitate, sorbitan monolaurate, sorbitan monooleate, sorbitan monopalmitate, sorbitan trioleate, sorbitan monostearate, sorbitan tristearate, polyoxyethylene (20) sorbitan monolaurate, polyoxyethylene (20) sorbitan monopalmitate, polyoxyethylene (20) sorbitan monostearate, isopropyl palmitate, isopropyl myristate, propylene glycol monolaurate, propylene glycol monocaprylate, or any other caprylate, caprate, laurate, linoleate, oleate, palmitate, stearate, isostearate.

18. The pharmaceutical composition according to claim 15, wherein the solvent is diethylene glycol monoethyl ether, octyl dodecanol, oleyl alcohol, dipropylene glycol, propylene glycol, or 1,2-butylene glycol.

19. The pharmaceutical composition according to claim 9, wherein the matrix polymer is a silicone adhesive and the permeation enhancer is diethylene glycol monoethyl ether.

20. The pharmaceutical composition according to claim 9, wherein the matrix polymer is hypromellose and the permeation enhancer is geraniol, and wherein the matrix layer further comprises polyethylene glycol as plasticiser.

21. The pharmaceutical composition according to claim 9, wherein the matrix layer further comprises a tackifier.

22. The pharmaceutical composition according to claim 9, wherein the matrix layer comprises
from 20 to 45 wt. % active ingredient;
from 30 to 70 wt. % matrix polymer(s);
from 2 to 20 wt. % permeation enhancer(s);
and optionally a plasticiser and/or tackifier.

23. The pharmaceutical composition according to claim 1, further comprising an occlusive backing layer.

24. A transdermal patch comprising a matrix layer, wherein the matrix layer comprises at least 5 wt. % of an active ingredient selected from aminophylline or theophylline or a salt thereof.

25. The transdermal patch according to claim 24, wherein the active ingredient is aminophylline.

26. A transdermal patch comprising:
(a) a backing layer;
(b) a matrix layer comprising aminophylline, a matrix polymer, a plasticizer, and a permeation enhancer,
(c) a removable protective release liner;
for use in the treatment or prevention of a hypomyelinating or a demyelinating disease or condition or a lesion of the peripheral or central nervous system where demyelination occurs.

27. Use of aminophylline or theophylline or a salt thereof for the manufacture of a medicament for treating or preventing a hypomyelinating or demyelinating disease or condition, wherein the medicament comprises a pharmaceutical composition in the form of a transdermal patch, and wherein the aminophylline or theophylline or salt thereof is the sole active ingredient in the transdermal patch.

28. A method of treating a subject affected with, or at risk of becoming affected with, a hypomyelinating or demyelinating disease or condition, the method comprising a step of administering a pharmaceutical composition comprising an active ingredient selected from aminophylline or theophylline or a salt thereof,
wherein the composition is in the form of a transdermal patch, and
wherein the aminophylline or theophylline or salt thereof is the sole active ingredient in the transdermal patch.
